(19) United States
(12) Patent Application Publication
(10) Pub. No.: US 2008/0241599 A1
(43) Pub. Date: Oct. 2, 2008

(54) CARBONIC ACID ESTER AND MAGNETIC RECORDING MEDIUM

(75) Inventors: Akira IMAKUNI, Odawara-shi (JP); Masahiko MORI, Odawara-shi (JP); Yoshihiro NAKAI, Odawara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/058,413

(22) Filed: Mar. 28, 2008

(30) Foreign Application Priority Data
Mar. 28, 2007 (JP) 2007-084968

(51) Int. Cl. G11B 5/66; C07C 69/96

(52) U.S. Cl. 428/829; 558/277

(57) ABSTRACT

A carbonic acid ester is provided that is represented by the formula below and has a melting point of no greater than 0° C.

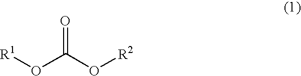

(In the formula, R¹ and R² independently denote a saturated hydrocarbon group, R¹ is a branched chain, and R² is a straight or branched chain). There is also provided a magnetic recording medium that includes a non-magnetic support and, above the support, at least one magnetic layer including a ferromagnetic powder dispersed in a binder, the magnetic layer including the carbonic acid ester. Furthermore, there is provided a magnetic recording medium including a support and, above the support, a non-magnetic layer including a non-magnetic powder dispersed in a binder, and above the non-magnetic layer, at least one magnetic layer including a ferromagnetic powder dispersed in a binder, the non-magnetic layer and/or the magnetic layer including the carbonic acid ester.

### BACKGROUND OF THE INVENTION

1. Field of the Invention

The present invention relates to a carbonic acid ester that can suitably be used as a lubricant, and a magnetic recording medium employing the carbonic acid ester as a lubricant.

2. Description of the Related Art

Magnetic recording technology has the excellent features, not seen in other recording methods, that the medium can be used repeatedly, signals are easily converted to electronic form and it is possible to build a system in combination with peripheral equipment, and signals can easily be corrected, and is therefore widely used in various fields including video, audio, and computer applications.

A magnetic recording medium that satisfies recent requirements for a larger recording capacity and a higher recording density has an extremely smooth surface in order to achieve high electromagnetic conversion characteristics. When a recording head slides against this smooth surface at high speed, it becomes very difficult to ensure durability by conventional techniques.

In order to improve the durability of a magnetic recording medium, for example, a magnetic recording medium employing a carbonate compound as a lubricant has been proposed (JP-A-7-138586 (JP-A denotes a Japanese unexamined patent application publication.) and JP-A-8-77547).

Furthermore, a magnetic recording medium having on the surface a specific abrasive protrusion density and having a specified acid hydrolysis rate has been proposed (JP-A-2003-323711).

### BRIEF SUMMARY OF THE INVENTION

The lubricant described in JP-A-7-138586 cannot exhibit a lubrication effect in a low temperature environment, and the durability cannot be sufficiently improved. Furthermore, the lubricants described in JP-A-8-77547 and JP-A-2003-323711 cause an oxidative fragmentation reaction, and sufficient storage stability cannot be obtained.

It is an object of the present invention to provide a carbonic acid ester that can be used suitably as a lubricant.

It is another object of the present invention to provide, using the carbonic acid ester, a magnetic recording medium having excellent durability in a low temperature environment and excellent storage stability.

### MEANS FOR SOLVING THE PROBLEMS

The objects of the present invention have been attained by means described in (1), (8), or (12), which are shown below together with (2) to (7), (9) to (11), (13), and (14), which are preferred embodiments.

(1) A carbonic acid ester represented by Formula (1) and having a melting point of no greater than 0° C.,

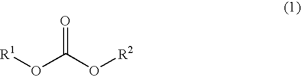

(in Formula (1), R¹ and R² independently denote a saturated hydrocarbon group, R¹ is a branched chain, and R² is a straight or branched chain),
(2) the carbonic acid ester according to (1) above, wherein the sum of the number of carbons of R¹ and R² is at least 12 but no greater than 50,
(3) the carbonic acid ester according to (1) or (2) above, wherein the number of carbons of R¹ is at least 3 but no greater than 12,
(4) the carbonic acid ester according to any one of (1) to (3) above, wherein R¹ has a structure in which it is branched at the β-position,
(5) the carbonic acid ester according to any one of (1) to (4) above, wherein R¹ is selected from the group consisting of a 2-methylpropyl group, a 2-methylbutyl group, and a 2-ethylhexyl group,
(6) the carbonic acid ester according to any one of (1) to (5) above, wherein R² is a straight-chain structure having at least 12 but no greater than 16 carbon atoms,
(7) the carbonic acid ester according to any one of (1) to (6) above, wherein R² is selected from the group consisting of an n-dodecyl group, an n-tetradecyl group, and an n-hexadecyl group,
(8) a magnetic recording medium comprising a non-magnetic support and, above the support, at least one magnetic layer comprising a ferromagnetic powder dispersed in a binder, the magnetic layer comprising the carbonic acid ester according to any one of (1) to (7) above,
(9) the magnetic recording medium according to (8) above, wherein the magnetic layer comprises at least 0.1 wt % but no greater than 5 wt % of the carbonic acid ester,
(10) the magnetic recording medium according to (8) or (9) above, wherein the non-magnetic support has a thickness of at least 3 μm but no greater than 80 μm,
(11) the magnetic recording medium according to any one of (8) to (10) above, wherein the magnetic layer has a thickness of at least 0.01 μm but no greater than 0.5 μm,
(12) a magnetic recording medium comprising a support and, above the support, a non-magnetic layer comprising a non-magnetic powder dispersed in a binder and, above the non-magnetic layer, at least one magnetic layer comprising a ferromagnetic powder dispersed in a binder, the non-magnetic layer and/or the magnetic layer comprising the carbonic acid ester according to any one of (1) to (7) above,
(13) the magnetic recording medium according to (12) above, wherein the non-magnetic layer comprises at least 0.1 wt % but no greater than 5 wt % of the carbonic acid ester, and
(14) the magnetic recording medium according to (12) or (13) above, wherein the non-magnetic layer has a thickness of at least 0.2 μm but no greater than 3.0 μm.

### DETAILED DESCRIPTION OF THE INVENTION

The carbonic acid ester of the present invention is represented by Formula (1) and has a melting point of no greater than 0° C.

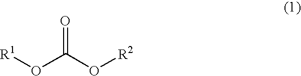

(In Formula (1), R¹ and R² independently denote a saturated hydrocarbon group, R¹ is a branched chain, and R² is a straight or branched chain.)

The carbonic acid ester of the present invention has a carbonate skeleton that is resistant to hydrolysis, and the carbonic acid ester represented by Formula (1) above has a low viscosity for its molecular weight and has a melting point of no greater than 0° C. Such a carbonic acid ester can be used suitably as a lubricant and can exhibit a sufficient lubrication effect even in a low temperature environment. In particular, even when the carbonic acid ester of the present invention is used as a lubricant for a medium whose surface is made to slide, since it has hydrolysis resistance and a low melting point, the storage stability is excellent, and a sufficient surfactant effect can be exhibited in a low temperature environment. The carbonic acid ester of the present invention can be used particularly suitably as a lubricant contained in the magnetic layer of a magnetic recording medium, thereby giving good transport durability in a low temperature environment and improving the storage stability.

In Formula (1), it is preferable for the sum of the number of carbons of R¹ and R² to be at least 12 but no greater than 50, more preferably at least 12 but no greater than 40, yet more preferably at least 12 but no greater than 30, and particularly preferably at least 16 but no greater than 24. It is preferable for the sum of the number of carbons of the two to be at least 12 since the volatility is low, and when used as a lubricant in the magnetic recording medium, there is little flying off from the surface of the magnetic layer during transport, and transport stoppage is not caused. It is also preferable for the sum of the number of carbons of the two to be no greater than 50 since the mobility of the molecule is high; when it is used as a lubricant in a magnetic recording medium a required amount of lubricant can exude to the surface, and it does not cause transport stoppage.

In Formula (1), R¹ denotes a saturated hydrocarbon group having a branched chain. If R¹ and R² have a straight chain structure, the melting point increases, and a sufficient lubrication effect cannot be obtained in a low temperature environment.

The number of carbons of R¹ is preferably at least 3 but no greater than 12, and more preferably at least 4 but no greater than 10. Furthermore, R¹ preferably has a structure in which it is branched at the β-position, and more preferably has a branched chain structure selected from the group consisting of a 2-methylpropyl group, a 2-methylbutyl group, and a 2-ethylhexyl group.

It is preferable for it to have a structure in which it is branched at the β-position since a carbonic acid ester having a low melting point can be obtained.

In Formula (1), R² denotes a saturated hydrocarbon group having a branched chain or a straight chain. R² preferably has a straight chain.

Examples of the saturated hydrocarbon group having a straight chain include a butyl group, a hexyl group, an octyl group, a decyl group, a dodecyl group, a tetradecyl group, a hexadecyl group, and an octadecyl group.

Among them, R² preferably has at least 8 but no greater than 20 carbons, R² more preferably has at least 10 but no greater than 18 carbons, and yet more preferably at least 12 but no greater than 16 carbons. Furthermore, from the viewpoint of availability of starting materials, R² more preferably has 12, 14, or 16 carbons. That is, R² is preferably selected from the group consisting of an n-dodecyl group, an n-tetradecyl group, and an n-hexadecyl group.

It is preferable for the number of carbons of R² to be at least 8 since from the viewpoint of molecular orientation a high lubrication effect is obtained. Furthermore, it is preferable for the number of carbons of R² to be at least 12 since the volatility is low, and when used in a magnetic recording medium, stable lubrication properties are shown. Moreover, it is preferable for the number of carbons of R² to be no greater than 16 since a melting point of no greater than 0° C. can be obtained.

A process for synthesizing the carbonic acid ester (carbonate) compound represented by Formula (1) of the present invention is not particularly limited, and a known carbonic acid ester synthetic process may be employed. Examples thereof include a process in which a chloroformate ester and an alcohol are reacted, a process in which a carbonic acid ester having a lower hydrocarbon group and an alcohol are reacted, a process in which a diaryl carbonic acid ester and an alcohol are reacted, a process in which carbon monoxide and an alcohol are reacted using a metal catalyst, and a process in which phosgene or a phosgene equivalent such as triphosgene and an alcohol are reacted. Among them, the process in which a chloroformic acid ester and an alcohol having a saturated hydrocarbon group are reacted is preferable since two different saturated hydrocarbon groups can easily be introduced and a single type of carbonic acid ester can be synthesized. The lower hydrocarbon group referred to here means a hydrocarbon group that has a smaller number of carbons than the saturated hydrocarbon group of the alcohol used in the reaction.

Specific examples of the chloroformic acid ester, which is a starting material for the synthetic reaction, include such as methyl chloroformate, ethyl chloroformate, butyl chloroformate, sec-butyl chloroformate, isobutyl chloroformate, propyl chloroformate, isopropyl chloroformate, 2-ethylhexyl chloroformate, 2-methylpropyl chloroformate, and 2-methylbutyl chloroformate.

The reaction temperature of the synthetic reaction is not particularly limited as long as the reaction proceeds, but is preferably in the range of at least 0° C. but no greater than 60° C. (hereinafter, ‘at least 0° C. but no greater than 60° C.’ is also written as ‘0° C. to 60° C.’ or ‘0 to 60° C.’, the same applies below), more preferably 0° C. to 40° C., and yet more preferably 0° C. to 25° C. The pressure may be a reduced pressure or normal pressure, and normal pressure conditions are preferable from the viewpoint of cost.

The synthetic reaction may employ a catalyst, and when a catalyst is used, it is preferably used at an equivalent amount of 0.001% to 1.0% relative to the carbonate reaction substrate of the chloroformic acid ester compound, carbonic acid ester having a lower hydrocarbon group or an aryl group, phosgene, etc., which are reaction starting materials.

Examples of such a catalyst include organic bases such as pyridine, N,N-dimethyl-4-aminopyridine, 2-methylpyridine, 4-methylpyridine, imidazole, N-methylimidazole, N-methylmorpholine, and benzotriazole, metal hydroxides such as lithium hydroxide, calcium hydroxide, and magnesium hydroxide, carbonates such as lithium carbonate, sodium carbonate, potassium carbonate, and cesium carbonate, and hydrogen carbonates such as sodium hydrogen carbonate and potassium hydrogen carbonate; an organic base that does not have an N—H bond when it is neutral, such as pyridine, N,N-dimethyl-4-aminopyridine, 2-methylpyridine, 4-methylpyridine, N-methylimidazole, or benzotriazole, or lithium hydroxide is preferable, and among them pyridine and derivatives thereof such as pyridine, N,N-dimethyl-4-aminopyridine, 2-methylpyridine, and 4-methylpyridine are more preferable.

As a method for taking out the carbonic acid ester (carbonate) compound represented by Formula (1) of the present invention from a reaction liquid, a separation method involving extraction, distillation, crystallization, etc. may be cited. From the viewpoint of industrial productivity, it is preferable to employ a method involving extraction, which causes little loss. Solvents used in the extraction are explained below.

Since the carbonic acid ester of the present invention has a high solubility in a saturated hydrocarbon-based solvent, as a solvent (extraction solvent) that undergoes phase separation from the saturated hydrocarbon solvent, a solvent comprising an organic solvent that is not infinitely miscible with the saturated hydrocarbon solvent is preferable. As an extraction method, it is preferable to carry out liquid-liquid extraction using the extraction solvent described above and a saturated hydrocarbon solvent. It is necessary for the solvent used in extraction to dissolve impurities, and in order to remove a base, etc. used in the reaction it is preferable to use a solvent that is infinitely miscible with water.

The saturated hydrocarbon solvent that can be used in the present invention is not particularly limited as long as it can dissolve the carbonic acid ester of the present invention, but from the viewpoint of ease of handling of the solvent and ease of a separation operation, a saturated hydrocarbon solvent having a boiling point of 35° C. to 85° C. is preferable, heptane, hexane, or a mixed solvent thereof is more preferable, and hexane is yet more preferable. Furthermore, the saturated hydrocarbon solvent may be used singly or as a mixture of two or more types in any proportions.

Moreover, since the alcohol used as a starting material for the carbonic acid ester of the present invention has extremely low solubility in water, there are cases in which it is necessary to remove as an impurity the alcohol remaining in the system as an unreacted component. Because of this, as an extraction solvent, a solvent comprising methanol, ethanol, propanol, acetonitrile, ethylene glycol and/or propylene glycol is preferable, and methanol and/or acetonitrile are more preferable.

In addition to use of the above-mentioned solvent on its own, it is possible to use a mixed solvent that can remove by-products and residual impurities from the saturated hydrocarbon solvent reaction system. Preferred specific examples thereof include a mixed solvent of methanol and water, a mixed solvent of acetonitrile and water, a mixed solvent of propylene glycol and water, and a mixed solvent of methanol and ethylene glycol.

### Magnetic Recording Medium

The carbonic acid ester of the present invention is used suitably as a lubricant of a magnetic recording medium.

The magnetic recording medium of the present invention comprises a non-magnetic support and, above the support, at least one magnetic layer comprising a ferromagnetic powder dispersed in a binder, the magnetic layer comprising the carbonic acid ester of the present invention.

Another magnetic recording medium of the present invention preferably comprises, between a non-magnetic support and a magnetic layer, a non-magnetic layer comprising a non-magnetic powder dispersed in a binder, the magnetic layer and/or the non-magnetic layer comprising the carbonic acid ester of the present invention.

In the present invention, the magnetic recording medium preferably has a non-magnetic layer, and the magnetic layer and the non-magnetic layer preferably comprise the carbonic acid ester of the present invention.

The magnetic layer formed above the non-magnetic support preferably comprises at least 0.1 wt % but no greater than 5.0 wt %, more preferably at least 0.5 wt % but no greater than 5 wt %, and yet more preferably at least 1 wt % but no greater than 3 wt % of the carbonic acid ester of the present invention.

Furthermore, the non-magnetic layer preferably comprises at least 0.1 wt % but no greater than 5 wt %, preferably at least 0.5 wt % but no greater than 5 wt %, and at least 1 wt % but no greater than 3 wt % of the carbonic acid ester of the present invention.

It is preferable for the content of the carbonic acid ester of the present invention in the magnetic layer and/or the non-magnetic layer to be in the above-mentioned range since a magnetic recording medium that gives a high lubrication effect and excellent durability in a low temperature environment and excellent storage stability can be obtained.

It is conventionally known that as an effect of a lubricant present on the surface of a magnetic layer, the sliding properties between a head and a tape are closely related to the amount of lubricant on the surface. A lubricant present on the surface of the magnetic layer in a stable state can reduce the sliding resistance between the head and the tape, thus improving the transport durability. Accompanying a recent demand for higher capacity of magnetic recording media, it is necessary to make the magnetic layer thinner; the amount of lubricant contained in the magnetic layer therefore becomes smaller due to the thinner magnetic layer, the lubricant is gradually removed by sliding against a recording/playback head, and due to an insufficient amount of lubricant scraping off might occur, thus causing stoppages, etc. Furthermore, in order to improve magnetic properties, it is necessary to make the surface of the magnetic layer more and more smooth, and because of this a conventional lubricant cannot exhibit a sufficient effect on transport properties, repetitive transport properties, and durability. When the amount of conventional lubricant is small, if the amount of lubricant is increased in order to enhance the lubrication effect, the mechanical strength of the magnetic coating is degraded, the magnetic layer is scraped off, and scraped-off powder might contaminate the transport path, or sufficient repetitive transport durability cannot be obtained.

Conventionally, a mixture of a fatty acid ester such as butyl stearate and a fatty acid such as myristic acid is used. However, when a fatty acid ester and a fatty acid are used, there is the problem that the friction increases during transport under high humidity conditions, and the transport tension of the magnetic tape becomes high.

When a fatty acid is used on its own, it is necessary to use a large amount thereof in order to obtain slipperiness, and in this case there are the defects that the magnetic layer becomes soft, the mechanical strength is degraded, and high speed sliding durability, which corresponds to the relative speed between the tape and the head, becomes poor. When a fatty acid and a fatty acid ester compound are used in combination, the high speed sliding durability becomes good and the tension becomes relatively low, but there is the defect that the transport tension becomes high under high humidity conditions such as 85% RH (relative humidity).

The present inventors have found that good transport durability can be guaranteed by using as a lubricant a carbonic acid ester (carbonate) having a saturated alkyl group represented by Formula (1) above. Since the carbonic acid ester of the present invention has a lower viscosity than expected for its molecular weight, its fluid lubrication properties are high, and its hydrolysis resistance is excellent and its storage stability is high since it is a carbonate and not a fatty acid ester.

JP-A-7-138586 discloses a magnetic recording medium employing a carbonate as a lubricant, but since it has a high melting point, the lubrication effect in a low temperature environment in particular was not sufficient. Since the carbonic acid ester of the present invention has a melting point of no greater than 0° C., an excellent lubrication effect can be exhibited even in a low temperature environment.

Furthermore, although JP-A-8-77547 discloses a magnetic recording medium employing an unsaturated alkyl carbonic acid ester, since this carbonic acid ester has an unsaturated group, it is oxidized and it is difficult to guarantee sufficient storage stability. The carbonic acid ester of the present invention, which does not contain an unsaturated bond, is not oxidized and can exhibit good storage stability.

Furthermore, JP-A-2003-323711 describes a magnetic recording medium employing a fatty acid ester, but a fatty acid ester is hydrolyzed and it is difficult to guarantee storage stability. The carbonic acid ester of the present invention has high resistance to hydrolysis and can give good storage stability.

### I. Magnetic Layer

The magnetic layer of the magnetic recording medium of the present invention is a layer comprising the carbonic acid ester of the present invention and comprising a ferromagnetic powder dispersed in a binder, and is a layer contributing to magnetic recording and playback.

### Ferromagnetic Metal Powder

The ferromagnetic powder used in the magnetic recording medium of the present invention is a cobalt-containing ferromagnetic iron oxide or ferromagnetic alloy powder, and the S_{BET} specific surface area is preferably 40 to 80 m²/g, and more preferably 50 to 70 m²/g. The crystallite size is preferably 12 to 25 nm, more preferably 13 to 22 nm, and particularly preferably 14 to 20 nm. The major axis length is preferably 0.05 to 0.25 μm, more preferably 0.07 to 0.2 μm, and particularly preferably 0.08 to 0.15 μm.

Examples of the ferromagnetic powder include yttrium-containing Fe, Fe—Co, Fe—Ni, and Co—Ni—Fe, and the yttrium content in the ferromagnetic powder is preferably 0.5 to 20 atom % as the yttrium atom/Fe atom ratio Y/Fe, and more preferably 5 to 10 atom %. When it is in such a range, the ferromagnetic powder has a high as value, and since the iron content is appropriate, the magnetic properties are good, and electromagnetic conversion characteristics are excellent. Furthermore, it is also possible for aluminum, silicon, sulfur, scandium, titanium, vanadium, chromium, manganese, copper, zinc, molybdenum, rhodium, palladium, tin, antimony, boron, barium, tantalum, tungsten, rhenium, gold, lead, phosphorus, lanthanum, cerium, praseodymium, neodymium, tellurium, bismuth, etc. to be present at 20 atom % or less relative to 100 atom % of iron. It is also possible for the ferromagnetic metal powder to contain a small amount of water, a hydroxide, or an oxide.

With regard to the magnetic recording medium of the present invention, one example of a process for producing the ferromagnetic powder into which cobalt or yttrium has been introduced is illustrated below. For example, an iron oxyhydroxide obtained by blowing an oxidizing gas into an aqueous suspension in which a ferrous salt and an alkali have been mixed can be used as a starting material. This iron oxyhydroxide is preferably of the α-FeOOH type, and with regard to a production process therefor, there is a first production process in which a ferrous salt is neutralized with an alkali hydroxide to form an aqueous suspension of Fe(OH)₂, and an oxidizing gas is blown into this suspension to give acicular α-FeOOH. There is also a second production process in which a ferrous salt is neutralized with an alkali carbonate to form an aqueous suspension of FeCO₃, and an oxidizing gas is blown into this suspension to give spindle-shaped α-FeOOH. Such an iron oxyhydroxide is preferably obtained by reacting an aqueous solution of a ferrous salt with an aqueous solution of an alkali to give an aqueous solution containing ferrous hydroxide, and then oxidizing this with air, etc. In this case, the aqueous solution of the ferrous salt may contain an Ni salt, a salt of an alkaline earth element such as Ca, Ba, or Sr, a Cr salt, a Zn salt, etc., and by selecting these salts appropriately the particle shape (axial ratio), etc. can be adjusted.

As the ferrous salt, ferrous chloride, ferrous sulfate, etc. are preferable. As the alkali, sodium hydroxide, aqueous ammonia, ammonium carbonate, sodium carbonate, etc. are preferable. With regard to salts that can be present at the same time, chlorides such as nickel chloride, calcium chloride, barium chloride, strontium chloride, chromium chloride, and zinc chloride are preferable. In a case where cobalt is subsequently introduced into the iron, before introducing yttrium, an aqueous solution of a cobalt compound such as cobalt sulfate or cobalt chloride is mixed and stirred with a slurry of the above-mentioned iron oxyhydroxide. After the slurry of iron oxyhydroxide containing cobalt is prepared, an aqueous solution containing a yttrium compound is added to this slurry, and they are stirred and mixed.

Neodymium, samarium, praseodymium, lanthanum, gadolinium, etc. can be introduced into the ferromagnetic powder used in the present invention as well as yttrium. They can be introduced using a chloride such as yttrium chloride, neodymium chloride, samarium chloride, praseodymium chloride, or lanthanum chloride or a nitrate salt such as neodymium nitrate or gadolinium nitrate, and they can be used in a combination of two or more types. The form of the ferromagnetic powder is not particularly limited, but acicular, granular, cubical, grain-shaped, or tabular form, etc. is normally employed. It is particularly preferable to use an acicular ferromagnetic powder.

As the ferromagnetic powder used in the magnetic layer of the present invention, a hexagonal ferrite powder may also be used.

Examples of the hexagonal ferrite include substitution products of barium ferrite, strontium ferrite, lead ferrite, and calcium ferrite, and Co substitution products. More specifically, magnetoplumbite type barium ferrite and strontium ferrite, magnetoplumbite type ferrite with a particle surface coated with a spinel, magnetoplumbite type barium ferrite and strontium ferrite partially containing a spinel phase, etc., can be cited. In addition to the designated atoms, an atom such as Al, Si, S, Sc, Ti, V, Cr, Cu, Y, Mo, Rh, Pd, Ag, Sn, Sb, Te, Ba, Ta, W, Re, Au, Hg, Pb, Bi, La, Ce, Pr, Nd, P, Co, Mn, Zn, Ni, Sr, B, Ge, Nb, or Zr may be included. For example, those to which Co—Ti, Co—Ti—Zr, Co—Ti—Zn, Ni—Ti—Zn, Nb—Zn—Co, Sb—Zn—Co, Nb—Zn, etc. have been added can be used. Characteristic impurities may be included depending on the starting material and the production process.

The particle size is preferably 10 to 200 nm as a hexagonal plate size, and more preferably 20 to 100 nm. When a magnetoresistive head is used for playback, the plate size is preferably 40 nm or less so as to reduce noise. When the plate size is in such a range, stable magnetization can be expected due to suppression of thermal fluctuations, and noise can be reduced.

The tabular ratio (plate size/plate thickness) is preferably 1 to 15, and more preferably 2 to 7. When the tabular ratio is in such a range, adequate orientation can be obtained, there is little inter-particle stacking, and noise can be suppressed. The specific surface area obtained by the BET method (S_{BET}) of a powder having a particle size within this range is usually 10 to 200 m²/g. The specific surface area substantially coincides with the value obtained by calculation using the plate size and the plate thickness.

The crystallite size is preferably 50 to 450 Å (5 to 45 nm), and more preferably 100 to 350 Å (10 to 35 nm). The plate size and the plate thickness distributions are preferably as narrow as possible. Although it is difficult, the distribution can be expressed using a numerical value by randomly measuring 500 particles on a transmission electron microscope (TEM) photograph of the particles. The distribution is not a regular distribution in many cases, but the standard deviation calculated with respect to the average size is σ/average size=0.1 to 2.0. In order to narrow the particle size distribution, the reaction system used for forming the particles is made as homogeneous as possible, and the particles so formed are subjected to a distribution-improving treatment. For example, a method of selectively dissolving ultrafine particles in an acid solution is also known.

The coercive force (Hc) measured for the magnetic substance can be adjusted so as to be on the order of 500 to 5,000 Oe (39.8 to 398 kA/m). A higher Hc is advantageous for high-density recording, but it is restricted by the capacity of the recording head. The coercive force Hc is preferably on the order of 800 to 4,000 Oe (63.7 to 318 kA/m), and more preferably at least 1,500 to 3,500 Oe (119.4 to 278.6 kA/m). When the saturation magnetization of the head exceeds 1.4 T, it is preferably 2,000 Oe or higher. The Hc can be controlled by the particle size (plate size, plate thickness), the type and amount of element included, the element replacement sites, the conditions used for the particle formation reaction, etc.

The saturation magnetization (σs) is preferably 40 to 80 emu/g (40 to 80 A·m²/kg). A higher σs is preferable, but there is a tendency for it to become lower when the particles become finer. In order to improve the σs, making a composite of magnetoplumbite ferrite with spinel ferrite, selecting the types of element included and their amount, etc. are well known. It is also possible to use a W type hexagonal ferrite.

When dispersing the magnetic substance, the surface of the magnetic substance can be treated with a material that is compatible with a dispersing medium and the polymer (binder). With regard to a surface-treatment agent, an inorganic or organic compound can be used. Representative examples include oxides and hydroxides of Si, Al, P, etc., and various types of silane coupling agents and various kinds of titanium coupling agents. The amount thereof is preferably 0.1% to 10% based on the magnetic substance.

The pH of the magnetic substance is also important for dispersion. It is usually on the order of 4 to 12, and although the optimum value depends on the dispersing medium and the polymer (binder), it is selected from on the order of 6 to 10 from the viewpoints of chemical stability and storage properties of the medium. The moisture contained in the magnetic substance also influences the dispersion. Although the optimum value depends on the dispersing medium and the polymer (binder), it is preferably 0.01% to 2.0%.

With regard to a production method for the hexagonal ferrite, there is glass crystallization method (1) in which barium oxide, iron oxide, a metal oxide that replaces iron, and boron oxide, etc. as glass forming materials are mixed so as to give a desired ferrite composition, then melted and rapidly cooled to give an amorphous substance, subsequently reheated, then washed and ground to give a barium ferrite crystal powder; hydrothermal reaction method (2) in which a barium ferrite composition metal salt solution is neutralized with an alkali, and after a by-product is removed, it is heated in a liquid phase at 100° C. or higher, then washed, dried and ground to give a barium ferrite crystal powder; co-precipitation method (3) in which a barium ferrite composition metal salt solution is neutralized with an alkali, and after a by-product is removed, it is dried and treated at 1,100° C. or less, and ground to give a barium ferrite crystal powder, etc., but any production method can be used in the present invention.

Furthermore, as a ferromagnetic powder that can be used in the magnetic layer of the magnetic recording medium of the present invention, iron nitride particles may also be used.

Iron nitride particles that can be used in the present invention are a spherical or ellipsoidal iron nitride-based magnetic substance having at least Fe and N as constituent elements. The ‘spherical’ referred to here means particles having a ratio of the maximum length to the minimum length of the particle diameter of at least 1 but less than 2, and the ‘ellipsoidal’ means particles having a ratio of the maximum length to the minimum length of the particle diameter of at least 2 but less than 4.

The spherical or ellipsoidal magnetic substance is preferably an iron nitride-based ferromagnetic powder containing Fe₁₆N₂ as a main phase. It may comprise, in addition to Fe and N atoms, an atom such as Al, Si, S, Sc, Ti, V, Cr, Cu, Y, Mo, Rh, Pd, Ag, Sn, Sb, Te, Ba, Ta, W, Re, Au, Hg, Pb, Bi, La, Ce, Pr, Nd, P, Co, Mn, Zn, Ni, Sr, B, Ge, or Nb. The content of N relative to Fe is preferably 1.0 to 20.0 atom %.

The iron nitride is preferably spherical or ellipsoidal, and the major axis length/minor axis length axial ratio is preferably 1 to 2. The BET specific surface area (S_{BET}) is preferably 30 to 100 m²/g, and more preferably 50 to 70 m²/g. The crystallite size is preferably 12 to 25 nm, and more preferably 13 to 22 nm. The saturation magnetization σs is preferably 50 to 200 A·m²/kg (emu/g), and more preferably 70 to 150 A·m²/kg (emu/g).

### Binder

In the present invention, a conventionally known thermoplastic resin, thermosetting resin, reactive resin or a mixture thereof is used as a binder of the magnetic layer.

The thermoplastic resin preferably has a glass transition temperature of −100° C. to 150° C., a number-average molecular weight of 1,000 to 200,000, and more preferably 10,000 to 100,000, and a degree of polymerization of 50 to 1,000.

Examples thereof include polymers and copolymers containing as a repeating unit vinyl chloride, vinyl acetate, vinyl alcohol, maleic acid, acrylic acid, an acrylate ester, vinylidene chloride, acrylonitrile, methacrylic acid, a methacrylate ester, styrene, butadiene, ethylene, vinyl butyral, vinyl acetal and vinyl ether; polyurethane resins; and various types of rubber resins.

Examples of the thermosetting resin and the reactive resin include phenol resins, epoxy resins, curable type polyurethane resins, urea resins, melamine resins, alkyd resins, reactive acrylic resins, formaldehyde resins, silicone resins, epoxy-polyamide resins, mixtures of a polyester resin and an isocyanate prepolymer, mixtures of a polyester polyol and a polyisocyanate, and mixtures of a polyurethane and a polyisocyanate.

Details of these resins are described in the ‘Purasuchikku Handobukku’ (Plastic Handbook) published by Asakura Shoten. It is also possible to use a known electron beam curable type resin in the non-magnetic layer (lower layer) or the magnetic layer (upper layer). Examples of the resin and a production method therefor are disclosed in detail in JP-A-62-256219. The above-mentioned resins can be used singly or in combination. Combinations of a polyurethane resin with at least one selected from a vinyl chloride resin, a vinyl chloride-vinyl acetate resin, a vinyl chloride-vinyl acetate-vinyl alcohol resin, a vinyl chloride-vinyl acetate-maleic anhydride copolymer, and nitrocellulose, and combinations thereof with a polyisocyanate are preferred.

Specific examples of the binder include VAGH, VYHH, VMCH, VAGF, VAGD, VROH, VYES, VYNC, VMCC, XYHL, XYSG, PKHH, PKHJ, PKHC and PKFE (manufactured by Union Carbide Corporation), MPR-TA, MPR-TA5, MPR-TAL, MPR-TSN, MPR-TMF, MPR-TS, and MPR-TM (manufactured by Nisshin Chemical Industry Co., Ltd.), 1000W, DX80, DX81, DX82, and DX83 (manufactured by Denki Kagaku Kogyo Kabushiki Kaisha), MR-110, MR-100, and 400X-110A (manufactured by Nippon Zeon Corporation), Nippollan N2301, N2302, and N2304 (manufactured by Nippon Polyurethane Industry Co., Ltd.), Pandex T-5105, T-R3080, and T-5201, Burnock D-400 and D-210-80, and Crisvon 6109 and 7209 (manufactured by Dainippon Ink and Chemicals, Incorporated), Vylon UR8200, UR8300, RV530, and RV280 (manufactured by Toyobo Co., Ltd.), Daiferamine 4020, 5020, 5100, 5300, 9020, 9022, and 7020 (manufactured by Dainichiseika Color & Chemicals Mfg. Co., Ltd.), MX5004 (manufactured by Mitsubishi Chemical Corp.), Sanprene SP-150 (manufactured by Sanyo Chemical Industries, Ltd.), and Saran F310 and F210 (manufactured by Asahi Kasei Corporation).

As the binder that can be used in the magnetic layer, among the above-mentioned binders, a vinyl chloride-based binder or a polyurethane-based binder is preferable, and a polyurethane containing a polar group and containing 3.5 mmol/g to 7 mmol/g of aromatic rings in the framework is particularly preferable.

Preferred examples of the polyurethane-based binder include polyester urethane, polyether urethane, polycarbonate urethane, polyether ester urethane, and acrylic polyurethane. The above-mentioned polyurethane-based binders are preferable since they have high affinity for the above-mentioned lubricant and the amount of surface lubricant can be controlled so as to be in an optimum range.

The polar group that the binder may have is preferably a sulfonate, a sulfamate, a sulfobetaine, a phosphate, a phosphonate, etc. The amount of polar group is preferably 1×10⁻⁵ eq/g to 2×10⁻⁴ eq/g.

The amount of binder, including curing agent, in the magnetic layer is preferably 10 to 25 parts by weight relative to 100 parts by weight of the ferromagnetic powder, the amount of binder in the non-magnetic lower layer, which will be described later is preferably 25 to 40 parts by weight relative to 100 parts by weight of the non-magnetic powder, and with regard to the amounts of binder in the magnetic layer and the non-magnetic lower layer, it is preferable to add a larger amount of binder to the non-magnetic layer.

It is possible to use the binder similar to the magnetic layer for the non-magnetic layer, in particular, the binder for the non-magnetic lower layer preferably has a framework containing a strongly polar group such as SO₃Na and a large number of aromatic groups. This enables the affinity between the lubricant and the non-magnetic layer binder to be increased, and allows a large amount of lubricant to be present in the non-magnetic layer in a stable manner.

When the affinity between the lubricant and the binder is appropriate, the binder and the lubricant are not completely miscible at the molecular level, and the lubricant can move to the upper layer (magnetic layer), which is preferable.

### Abrasive

The magnetic layer of the magnetic recording medium of the present invention preferably contains an abrasive.

An inorganic non-magnetic powder can be used as the abrasive. Examples of the inorganic non-magnetic powder include inorganic compounds such as a metal oxide, a metal carbonate, a metal sulfate, a metal nitride, a metal carbide, and a metal sulfide. As the inorganic compound, α-alumina with an α-component proportion of 90% to 100%, β-alumina, γ-alumina, silicon carbide, chromium oxide, cerium oxide, α-iron oxide, corundum, silicon nitride, titanium carbide, titanium oxide, silicon dioxide, tin oxide, magnesium oxide, tungsten oxide, zirconium oxide, boron nitride, zinc oxide, barium sulfate, molybdenum disulfide, etc. can be used singly or in combination. Particularly preferred are α-alumina, α-iron oxide (colcothar), and chromium oxide.

The abrasive that can be used in the present invention is used by varying the type, amount, particle size, combination, shape, etc.

When only one type of abrasive is used, the average particle size of the abrasive used in the present invention is preferably 0.05 to 0.4 μm, and more preferably 0.1 to 0.3 μm. It is preferable that particles with a particle size larger than the average particle size by 0.1 μm or more are present at a proportion of 1 to 40%, more preferably 5 to 30%, and most preferably 10 to 20%. Although the particle size of the abrasive itself affects the particle size of abrasive particles that are actually present on the surface of the magnetic layer, they are not equal to each other. The particle size of the abrasive particles present on the surface of the magnetic layer varies according to the dispersion conditions, etc. for the abrasive. Furthermore, some particles come out easily to the surface of the magnetic layer during coating and drying steps whereas it is difficult for others to come out to the surface.

Two or more abrasives having different average particle sizes may be used in combination. In this case, taking the weighted average value as the average particle size, which depends on the actual proportions used of the two or more abrasives, the particles with the average particle size and the particles with a particle size 0.1 μm or more greater than the average particle size can be set so as to be within the above-mentioned ranges.

Changing the dispersion conditions for the two abrasives can also control the particle size. For example, abrasive A is dispersed with a binder and a solvent in advance. This dispersion and abrasive B as a powder are added to a kneaded ferromagnetic metal powder that has been kneaded separately with a binder and a solvent, and the mixture is dispersed. In this way, the dispersion conditions for the abrasive A and the abrasive B can be varied. That is, the abrasive A is dispersed more strongly than the abrasive B. The tap density of the abrasive powder is preferably 0.05 to 2 g/mL, and more preferably 0.2 to 1.5 g/mL.

The water content of the abrasive powder is preferably 0.05 to 5 wt %, and more preferably 0.2 to 3 wt %. The specific surface area of the abrasive is preferably 1 to 100 m²/g, and more preferably 5 to 50 m²/g. Its oil absorption determined using DBP (dibutyl phthalate) is preferably 5 to 100 mL/100 g, and more preferably 10 to 80 mL/100 g. The specific gravity is preferably 1 to 12, and more preferably 3 to 6. The shape of the abrasive may be any one of acicular, spherical, polyhedral, and tabular. The surface of the abrasive may be coated at least partially with a compound which is different from the main component of the abrasive. Examples of the compound include Al₂O₃, SiO₂, TiO₂, ZrO₂, SnO₂, Sb₂O₃, and ZnO. In particular, the use of Al₂O₃, SiO₂, TiO₂ or ZrO₂ gives good dispersibility. These compounds may be used singly or in combination.

Specific examples of the abrasive that can be used in the magnetic layer of the present invention include Nanotite (manufactured by Showa Denko K.K.), Hit 100, Hit 82, Hit 80, Hit 70, Hit 60A, Hit 55, AKP-20, AKP-30, AKP-50, and ZA-G1 (manufactured by Sumitomo Chemical Co., Ltd.), ERC-DBM, HP-DBM, HPF-DBM, HPFX-DBM, HPS-DBM, and HPSX-DBM (manufactured by Reynolds Corp.), WA8000 and WA10000 (manufactured by Fujimi Incorporated), UB20, UB40B, and Mecanox UA (manufactured by C. Uyemura & Co., Ltd.), UA2055, UA5155, and UA5305 (manufactured by Showa Keikinzoku K.K.), G-5, Kromex M, Kromex S1, Kromex U2, Kromex U1, Kromex X10, and Kromex KX10 (manufactured by Nippon Chemical Industry Co., Ltd.), ND803, ND802, and ND801 (manufactured by Nippon Denko Co., Ltd.), F-1, F-2, and UF-500 (manufactured by Tosoh Corporation), DPN-250, DPN-250BX, DPN-245, DPN-270BX, TF-100, TF-120, TF-140, DPN-550BX, and TF-180 (manufactured by Toda Kogyo Corp.), A-3 and B-3 (manufactured by Showa Mining Co., Ltd.), beta SiC and UF (manufactured by Central Glass Co., Ltd.), β-Random Standard and β-Random Ultrafine (manufactured by Ibiden Co., Ltd.), JR401, MT-100S, MT-100T, MT-150W, MT-500B, MT-600B, MT-100F, and MT-500HD (manufactured by Tayca Corporation), TY-50, TTO-51B, TTO-55A, TTO-55B, TTO-55C, TTO-55S, TTO-55D, SN-100, E270, and E271 (manufactured by Ishihara Sangyo Kaisha Ltd.), STT-4D, STT-30D, STT-30, STT-65C, and Y-LOP, and calcined products thereof (manufactured by Titan Kogyo Kabushiki Kaisha), FINEX-25, BF-1, BF-10, BF-20, and ST-M (manufactured by Sakai Chemical Industry Co., Ltd.), HZn and HZr3M (manufactured by Hokkai Kagaku), DEFIC-Y and DEFIC-R (manufactured by Dowa Mining Co., Ltd.), AS2BM and TiO₂P25 (manufactured by Nippon Aerosil Co., Ltd.), and 100A and 500A (manufactured by Ube Industries, Ltd.).

### Additives

The magnetic layer of the magnetic recording medium of the present invention can comprise an additive as necessary. Examples of the additive include a dispersant/dispersion adjuvant, a fungicide, an antistatic agent, an antioxidant, a solvent, and carbon black. Furthermore, a lubricant other than the above-mentioned carbonic acid ester may be used in combination as an additive.

Examples of these additives include tungsten disulfide, graphite, graphite fluoride, a silicone oil, a polar group-containing silicone, a fatty acid-modified silicone, a fluorine-containing silicone, a fluorine-containing alcohol, a fluorine-containing ester, a polyolefin, a polyglycol, a polyphenyl ether; aromatic ring-containing organic phosphonic acids such as phenylphosphonic acid, benzylphosphonic acid, phenethylphosphonic acid, α-methylbenzylphosphonic acid, 1-methyl-1-phenethylphosphonic acid, diphenylmethylphosphonic acid, biphenylphosphonic acid, benzylphenylphosphonic acid, α-cumylphosphonic acid, tolylphosphonic acid, xylylphosphonic acid, ethylphenylphosphonic acid, cumenylphosphonic acid, propylphenylphosphonic acid, butylphenylphosphonic acid, heptylphenylphosphonic acid, octylphenylphosphonic acid, and nonylphenylphosphonic acid, and alkali metal salts thereof; alkylphosphonic acids such as octylphosphonic acid, 2-ethylhexylphosphonic acid, isooctylphosphonic acid, isononylphosphonic acid, isodecylphosphonic acid, isoundecylphosphonic acid, isododecylphosphonic acid, isohexadecylphosphonic acid, isooctadecylphosphonic acid, and isoeicosylphosphonic acid, and alkali metal salts thereof; aromatic phosphates such as phenyl phosphate, benzyl phosphate, phenethyl phosphate, α-methylbenzyl phosphate, 1-methyl-1-phenethyl phosphate, diphenylmethyl phosphate, biphenyl phosphate, benzylphenyl phosphate, α-cumyl phosphate, tolyl phosphate, xylyl phosphate, ethylphenyl phosphate, cumenyl phosphate, propylphenyl phosphate, butylphenyl phosphate, heptylphenyl phosphate, octylphenyl phosphate, and nonylphenyl phosphate, and alkali metal salts thereof; alkyl phosphates such as octyl phosphate, 2-ethylhexyl phosphate, isooctyl phosphate, isononyl phosphate, isodecyl phosphate, isoundecyl phosphate, isododecyl phosphate, isohexadecyl phosphate, isooctadecyl phosphate, and isoeicosyl phosphate, and alkali metal salts thereof; alkyl sulfonates and alkali metal salts thereof; fluorine-containing alkyl sulfates and alkali metal salts thereof; monobasic fatty acids that have 10 to 24 carbons, may contain an unsaturated bond, and may be branched, such as lauric acid, myristic acid, palmitic acid, stearic acid, behenic acid, oleic acid, linoleic acid, linolenic acid, elaidic acid, and erucic acid, and metal salts thereof; mono-fatty acid esters, di-fatty acid esters, and poly-fatty acid esters such as butyl stearate, octyl stearate, amyl stearate, isooctyl stearate, octyl myristate, butyl laurate, butoxyethyl stearate, anhydrosorbitan monostearate, anhydrosorbitan distearate, and anhydrosorbitan tristearate that are formed from a monobasic fatty acid that has 10 to 24 carbons, may contain an unsaturated bond, and may be branched, and any one of a mono- to hexa-hydric alcohol that has 2 to 22 carbons, may contain an unsaturated bond, and may be branched, an alkoxy alcohol that has 12 to 22 carbons, may have an unsaturated bond, and may be branched, and a mono alkyl ether of an alkylene oxide polymer; fatty acid amides having 2 to 22 carbons; aliphatic amines having 8 to 22 carbons; etc. Other than the above-mentioned hydrocarbon groups, those having an alkyl, aryl, or aralkyl group that is substituted with a group other than a hydrocarbon group, such as a nitro group, F, Cl, Br, or a halogen-containing hydrocarbon such as CF₃, CCl₃, or CBr₃ can also be used.

Furthermore, there are a nonionic surfactant such as an alkylene oxide type, a glycerol type, a glycidol type, or an alkylphenol-ethylene oxide adduct; a cationic surfactant such as a cyclic amine, an ester amide, a quaternary ammonium salt, a hydantoin derivative, a heterocyclic compound, a phosphonium salt, or a sulfonium salt; an anionic surfactant containing an acidic group such as a carboxylic acid, a sulfonic acid, or a sulfate ester group; and an amphoteric surfactant such as an amino acid, an aminosulfonic acid, a sulfate ester or a phosphate ester of an amino alcohol, or an alkylbetaine. Details of these surfactants are described in ‘Kaimenkasseizai Binran’ (Surfactant Handbook) (published by Sangyo Tosho Publishing).

The additives such as these dispersants and the lubricants used in combination need not always be pure and may contain, in addition to the main component, an impurity such as an isomer, an unreacted material, a by-product, a decomposition product, or an oxide. However, the impurity content is preferably 30 wt % or less, and more preferably 10 wt % or less.

Specific examples of these additives include NAA-102, hardened castor oil fatty acid, NAA-42, Cation SA, Nymeen L-201, Nonion E-208, Anon BF, and Anon LG (produced by Nippon Oil & Fats Co., Ltd.), FAL-205 and FAL-123 (produced by Takemoto Oil & Fat Co., Ltd), Enujelv OL (produced by New Japan Chemical Co., Ltd.), TA-3 (produced by Shin-Etsu Chemical Industry Co., Ltd.), Amide P (produced by Lion Armour), Duomin TDO (produced by Lion Corporation), BA-41G (produced by The Nisshin Oil Mills, Ltd.), and Profan 2012E, Newpol PE 61, and lonet MS-400 (produced by Sanyo Chemical Industries, Ltd.).

An organic solvent used for the magnetic layer of the magnetic recording medium of the present invention can be a known organic solvent. As the organic solvent, a ketone such as acetone, methyl ethyl ketone, methyl isobutyl ketone, diisobutyl ketone, cyclohexanone, or isophorone, an alcohol such as methanol, ethanol, propanol, butanol, isobutyl alcohol, isopropyl alcohol, or methylcyclohexanol, an ester such as methyl acetate, butyl acetate, isobutyl acetate, isopropyl acetate, ethyl lactate, or glycol acetate, a glycol ether such as glycol dimethyl ether, glycol monoethyl ether, or dioxane, an aromatic hydrocarbon such as benzene, toluene, xylene, or cresol, a chlorohydrocarbon such as methylene chloride, ethylene chloride, carbon tetrachloride, chloroform, ethylene chlorohydrin, chlorobenzene, or dichlorobenzene, N,N-dimethylformamide, hexane, tetrahydrofuran, etc. can be used at any ratio.

These organic solvents do not always need to be 100% pure, and may contain an impurity such as an isomer, an unreacted compound, a by-product, a decomposition product, an oxide, or moisture in addition to the main component. The content of these impurities is preferably 30% or less, and more preferably 10% or less. The organic solvent used in the present invention is preferably the same type for both the magnetic layer and the non-magnetic layer. However, the amount added may be varied. The coating stability is improved by using a high surface tension solvent (cyclohexanone, dioxane, etc.) for the non-magnetic layer; more specifically, it is important that the arithmetic mean value of the surface tension of the upper layer (magnetic layer) solvent composition is not less than that for the surface tension of the non-magnetic layer solvent composition. In order to improve the dispersibility, it is preferable for the polarity to be somewhat strong, and the solvent composition preferably contains 50% or more of a solvent having a permittivity of 15 or higher. The solubility parameter is preferably 8 to 11.

These dispersants and surfactants used in the magnetic layer of the magnetic recording medium of the present invention may be selected as necessary in terms of the type and amount according to the magnetic layer and the non-magnetic layer, which will be described later.

Furthermore, it is though that, for example, by adjusting the amount of surfactant the coating stability is improved. All or a part of the additives used in the present invention may be added to a magnetic coating solution or a non-magnetic coating solution at any stage of its preparation. For example, the additives may be blended with a ferromagnetic powder prior to a kneading step, they may be added in a step of kneading a ferromagnetic powder, a binder, and a solvent, they may be added in a dispersing step, they may be added after dispersion, or they may be added immediately prior to coating.

The magnetic layer of the magnetic recording medium of the present invention can contain as necessary carbon black.

Types of carbon black that can be used include furnace black for rubber, thermal black for rubber, black for coloring, and acetylene black. The carbon black should have characteristics that have been optimized as follows according to a desired effect, and the effect can be increased by the use thereof in combination.

The specific surface area of the carbon black is preferably 100 to 500 m²/g, and more preferably 150 to 400 m²/g, and the DBP oil absorption is preferably 20 to 400 mL/100 g, and more preferably 30 to 200 mL/100 g. The particle size of the carbon black is preferably 5 to 80 nm, more preferably 10 to 50 nm, and yet more preferably 10 to 40 nm. The pH of the carbon black is preferably 2 to 10, the water content thereof is preferably 0.1% to 10%, and the tap density is preferably 0.1 to 1 g/mL.

Specific examples of the carbon black that can be used in the present invention include BLACKPEARLS 2000, 1300, 1000, 900, 800, 880 and 700, and VULCAN XC-72 (manufactured by Cabot Corporation), #3050B, #3150B, #3250B, #3750B, #3950B, #950, #650B, #970B, #850B, MA-600, MA-230, #4000, and #4010 (manufactured by Mitsubishi Chemical Corporation), CONDUCTEX SC, and RAVEN 8800, 8000, 7000, 5750, 5250, 3500, 2100, 2000, 1800, 1500, 1255, and 1250 (manufactured by Columbian Carbon Co.), Ketjen Black EC (manufactured by Akzo), and Ketjen Black EC (manufactured by Ketjen Black International Company Ltd.).

The carbon black may be subjected to any of a surface treatment with a dispersant, etc., grafting with a resin, or a partial surface graphitization. The carbon black may also be dispersed in a binder prior to addition to a coating solution. The carbon black that can be used in the present invention can be selected by referring to, for example, the ‘Kabon Burakku Binran’ (Carbon Black Handbook) (edited by the Carbon Black Association of Japan).

The carbon black may be used singly or in a combination of different types thereof. When the carbon black is used, it is preferably used in an amount of 0.1 to 30 wt % based on the weight of the magnetic substance. The carbon black has the functions of preventing static charging of the magnetic layer, reducing the coefficient of friction, imparting light-shielding properties, and improving the film strength. Such functions vary depending upon the type of carbon black. Accordingly, it is of course possible in the present invention to appropriately choose the type, the amount and the combination of carbon black for the magnetic layer according to the intended purpose on the basis of the above mentioned various properties such as the particle size, the oil absorption, the electrical conductivity, and the pH value, and it is better if they are optimized for the respective layers.

### II. Non-Magnetic Layer

The non-magnetic layer is now explained in detail.

The magnetic recording medium of the present invention may comprise, between the non-magnetic support and the magnetic layer, at least one non-magnetic layer comprising a non-magnetic powder dispersed in a binder.

The binder is preferably the same binder as that of the magnetic layer.

### Non-Magnetic Powder

The non-magnetic powder used in the non-magnetic layer may be an inorganic material or an organic material. The non-magnetic layer may contain, together with the non-magnetic powder, carbon black as necessary.

The inorganic powder used in the lower coated layer is a non-magnetic powder, and may be selected from, for example, inorganic compounds such as a metal oxide, a metal carbonate, a metal sulfate, a metal nitride, a metal carbide, and a metal sulfide.

With regard to the inorganic compounds, for example, α-alumina with an a component proportion of at least 90%, β-alumina, γ-alumina, θ-alumina, silicon carbide, chromium oxide, cerium oxide, α-iron oxide, goethite, corundum, silicon nitride, titanium carbide, titanium oxide, silicon dioxide, tin oxide, magnesium oxide, tungsten oxide, zirconium oxide, boron nitride, zinc oxide, calcium carbonate, calcium sulfate, barium sulfate, molybdenum disulfide, etc. can be used singly or in combination. From the viewpoint of a narrow particle size distribution, the possibility of having many means for imparting functionality, etc., titanium dioxide, zinc oxide, iron oxide, and barium sulfate are particularly preferable, and titanium dioxide and α-iron oxide are more preferable.

The particle size of such a non-magnetic powder is preferably 0.005 to 2 μm, but it is also possible, as necessary, to combine non-magnetic powders having different particle sizes or widen the particle size distribution of a single non-magnetic powder, thus producing the same effect. The particle size of the non-magnetic powder is particularly preferably 0.01 to 0.2 μm. In particular, when the non-magnetic powder is a granular metal oxide, the average particle size is preferably 0.08 μm or less, and when it is an acicular metal oxide, the major axis length is preferably 0.3 μm or less. The tap density is preferably 0.05 to 2 g/mL, and more preferably 0.2 to 1.5 g/mL. The water content of the non-magnetic powder is preferably 0.1 to 5 wt %, more preferably 0.2 to 3 wt %, and yet more preferably 0.3 to 1.5 wt %. The pH of the non-magnetic powder is 2 to 11, and is particularly preferably in the range of 5.5 to 10. The specific surface area of the non-magnetic powder is preferably 1 to 100 m²/g, more preferably 5 to 80 m²/g, and yet more preferably 10 to 70 m²/g. The crystallite size of the non-magnetic powder is preferably 0.004 to 1 μm, and more preferably 0.04 to 0.1 μm. The oil absorption measured using DBP (dibutyl phthalate) is preferably 5 to 100 mL/100 g, more preferably 10 to 80 mL/100 g, and yet more preferably 20 to 60 mL/100 g. The specific gravity is preferably 1 to 12, and more preferably 3 to 6. The form may be any one of acicular, spherical, polyhedral, and tabular.

The ignition loss is preferably 20 wt % or less, and it is most preferable that there is no ignition loss. The Mohs hardness of the non-magnetic powder used in the present invention is preferably at least 4 but no greater than 10. The roughness factor of the surface of the powder is preferably 0.8 to 1.5, and more preferably 0.9 to 1.2. The amount of SA (stearic acid) absorbed by the non-magnetic powder is preferably 1 to 20 μmol/m², more preferably 2 to 15 μmol/m², and yet more preferably 3 to 8 μmol/m². The heat of wetting of the non-magnetic powder in water at 25° C. is preferably in the range of 200 to 600 erg/cm² (20 to 60 μJ/cm²). A solvent that gives a heat of wetting in this range can be used. The pH is preferably between 3 and 6. Water-soluble Na in the non-magnetic powder is preferably 0 to 150 ppm, and water-soluble Ca is preferably 0 to 50 ppm.

The surface of the non-magnetic powder is preferably subjected to a surface treatment so that Al₂O₃, SiO₂, TiO₂, ZrO₂, SnO₂, Sb₂O₃, ZnO, or Y₂O₃ is present. In terms of dispersibility in particular, Al₂O₃, SiO₂, TiO₂, and ZrO₂ are preferable, and Al₂O₃, SiO₂, and ZrO₂ are more preferable. They may be used in combination or singly. Depending on the intended purpose, a surface-treated layer may be obtained by co-precipitation, or a method in which it is firstly treated with alumina and the surface thereof is then treated with silica, or vice versa, can be employed. The surface-treated layer may be formed as a porous layer depending on the intended purpose, but it is generally preferable for it to be uniform and dense.

Specific examples of the non-magnetic powder used in the lower coated layer of the magnetic recording medium of the present invention include Nanotite (manufactured by Showa Denko K.K.), HIT-100 and ZA-G1 (manufactured by Sumitomo Chemical Co., Ltd.), α-hematite DPN-250, DPN-250BX, DPN-245, DPN-270BX, DBN-SA1, and DBN-SA3 (manufactured by Toda Kogyo Corp.), titanium oxide TTO-51B, TTO-55A, TTO-55B, TTO-55C, TTO-55S, TTO-55D, and SN-100, and α-hematite E270, E271, E300, and E303 (manufactured by Ishihara Sangyo Kaisha Ltd.), titanium oxide STT-4D, STT-30D, STT-30, and STT-65C, and α-hematite α-40 (manufactured by Titan Kogyo Kabushiki Kaisha), MT-100S, MT-100T, MT-150W, MT-500B, MT-600B, MT-100F, and MT-500HD (manufactured by Tayca Corporation), FINEX-25, BF-1, BF-10, BF-20, and ST-M (manufactured by Sakai Chemical Industry Co., Ltd.), DEFIC-Y and DEFIC-R (manufactured by Dowa Mining Co., Ltd.), AS2BM and TiO₂P25 (manufactured by Nippon Aerosil Co., Ltd.), 100A and 500A (manufactured by Ube Industries, Ltd.), and calcined products thereof.

Particularly preferred non-magnetic powders are titanium dioxide and α-iron oxide.

α-Iron oxide (hematite) is employed under the various conditions below. That is, with regard to the α-Fe₂O₃ powder that can be used in the present invention, its precursor particles are acicular goethite particles obtained by, for example, a normal method (1) for forming acicular goethite particles in which a ferrous hydroxide colloid-containing suspension obtained by adding at least an equivalent amount of an aqueous solution of an alkali hydroxide to an aqueous ferrous solution is subjected to an oxidation reaction at a pH of 11 or higher at a temperature of 80° C. or less while passing an oxygen-containing gas therethrough, a method (2) for forming spindle-shaped goethite particles in which an oxidation reaction is carried out by passing an oxygen-containing gas into a suspension containing FeCO₃ obtained by reacting an aqueous solution of a ferrous salt and an aqueous solution of an alkali carbonate, a method (3) for forming acicular goethite nuclei particles by carrying out an oxidation reaction by passing an oxygen-containing gas into an aqueous solution of a ferrous salt containing a ferrous hydroxide colloid obtained by adding less than an equivalent amount of an aqueous solution of an alkali hydroxide or an alkali carbonate to an aqueous solution of a ferrous salt, and subsequently growing the acicular goethite nuclei particles by adding an aqueous solution of an alkali hydroxide to the aqueous solution of the ferrous salt containing the acicular goethite nuclei particles in an amount that is at least equivalent to the Fe²⁺ in the aqueous solution of the ferrous salt, and then passing through an oxygen-containing gas, and a method (4) for forming acicular goethite nuclei particles by carrying out an oxidation reaction by passing an oxygen-containing gas into an aqueous solution of a ferrous salt containing a ferrous hydroxide colloid obtained by adding less than an equivalent amount of an aqueous solution of an alkali hydroxide or an alkali carbonate to an aqueous ferrous solution, and subsequently growing the acicular goethite nuclei particles in an acidic to neutral region.

During the reaction to form goethite particles, different types of elements such as Ni, Zn, P, and Si, which are normally added in order to improve the characteristics of the powder, etc., may be added without any problem. The acicular goethite particles, which are the precursor particles, are dehydrated at a temperature in the range of 200° C. to 500° C., and if necessary further annealed by heating at a temperature in the range of 350° C. to 800° C. to give acicular α-Fe₂O₃ particles. An anti-sintering agent such as P, Si, B, Zr, or Sb can be attached without problem to the surface of the acicular goethite particles that are to be dehydrated or annealed. Annealing by heating at a temperature in the range of 350° C. to 800° C. is carried out for blocking pores formed on the surface of the dehydrated acicular α-Fe₂O₃ particles by melting the very surface of the particles, thus giving a smooth surface configuration, which is preferable.

The α-Fe₂O₃ powder used in the present invention is obtained by subjecting the dehydrated or annealed acicular α-Fe₂O₃ particles to dispersion in an aqueous solution to give a suspension, coating the surface of the α-Fe₂O₃ particles with an Al compound by adding the compound and adjusting the pH, and further subjecting the particles to filtration, washing with water, drying, grinding, and if necessary further degassing/compacting, etc.

As the Al compound used, an aluminum salt such as aluminum acetate, aluminum sulfate, aluminum chloride, or aluminum nitrate or an alkali aluminate such as sodium aluminate can be used.

In this case, the amount of Al compound added on an Al basis is preferably 0.01 to 50 wt % relative to the α-Fe₂O₃ powder. When the amount of Al compound added is in the above-mentioned range, the dispersibility thereof in a binder resin is sufficient, there are few Al compounds suspended on the particle surface, and Al compounds do not interact, which is preferable.

With regard to the non-magnetic powder of the lower layer in the present invention, the coating can be carried out using, in addition to the Al compound, one or more types of compounds chosen from an Si compound, and P, Ti, Mn, Ni, Zn, Zr, Sn, and Sb compounds. The amount of these compounds, which are used together with the Al compound, is preferably in the range of 0.01 to 50 wt % relative to the α-Fe₂O₃ powder. When the amount added is in the above-mentioned range, the effect of improving the dispersibility by the addition is sufficient, there are few suspended compounds that are not on the particle surface, and the compounds do not interact, which is preferable.

Methods for producing titanium dioxide are as follows. The main methods for producing titanium oxide are a sulfuric acid method and a chlorine method. In the sulfuric acid method, an ilmenite ore is digested with sulfuric acid, and Ti, Fe, etc. are extracted as sulfates. Iron sulfate is removed by crystallization, and the remaining titanyl sulfate solution is purified by filtration and then subjected to thermal hydrolysis so as to precipitate hydrated titanium oxide. After this is filtered and washed, impurities are removed by washing, a particle size regulator, etc. is added thereto, and the mixture is calcined at 80° C. to 1,000° C. to give crude titanium oxide. The rutile type and the anatase type can be separated according to the type of a nucleating agent that is added when carrying out hydrolysis. This crude titanium oxide is subjected to grinding, size adjustment, surface treatment, etc. As an ore for the chlorine method, natural rutile or synthetic rutile is used. The ore is chlorinated at high temperature under reducing conditions, Ti is converted into TiCl₄ and Fe is converted into FeCl₂, and iron oxide solidifies by cooling and is separated from liquid TiCl₄. The crude TiCl₄ thus obtained is purified by distillation, then a nucleating agent is added, and the mixture is reacted momentarily with oxygen at a temperature of 1,000° C. or higher to give crude titanium oxide. A finishing method for imparting pigmentary properties to the crude titanium oxide formed by this oxidative decomposition process is the same as that for the sulfuric acid method.

The surface treatment is carried out by dry-grinding the above-mentioned titanium oxide material, then adding water and a dispersant thereto, and subjecting it to rough classification by wet-grinding and centrifugation. Subsequently, the fine grain slurry is transferred to a surface treatment vessel, and here surface coating with a metal hydroxide is carried out. Firstly, a predetermined amount of an aqueous solution of a salt such as Al, Si, Ti, Zr, Sb, Sn, or Zn is added, an acid or an alkali for neutralizing this is added, and the hydrated oxide thus formed is used for coating the surface of the titanium oxide particles. Water-soluble salts produced as a by-product are removed by decantation, filtration, and washing. Finally the pH of the slurry is adjusted, and it is filtered and washed with pure water. The cake thus washed is dried by a spray dryer or a band dryer. This dried product is ground using a jet mill to give a final product.

In addition to the an aqueous system, it is also possible to expose a titanium oxide powder to AlCl₃ or SiCl₄ vapor and then to steam, thereby carrying out a surface treatment with Al or Si. Other methods for preparing a pigment can be referred to in G. D. Parfitt and K. S. W. Sing, ‘Characterization of Powder Surfaces’ Academic Press, 1976.

Incorporation of carbon black into the non-magnetic layer can give the known effects of a lowering of surface electrical resistance (Rs), a reduction in light transmittance, and giving a desired micro Vickers hardness. The presence of carbon black in the lower layer can exhibit an effect of storing a lubricant. Types of carbon black that can be used include furnace black for rubber, thermal black for rubber, black for coloring, and acetylene black. The carbon black used in the non-magnetic layer should have characteristics that have been optimized as follows according to a desired effect, and the effect can be increased by the use thereof in combination.

The specific surface area of the carbon black in the non-magnetic layer is preferably 100 to 500 m²/g, and more preferably 150 to 400 m²/g, and the DBP oil absorption thereof is preferably 20 to 400 mL/100 g, and more preferably 30 to 200 mL/100 g. The particle size of the carbon black is preferably 5 to 80 nm, more preferably 10 to 50 nm, and yet more preferably 10 to 40 nm. The pH of the carbon black is preferably 2 to 10, the water content is preferably 0.1% to 10%, and the tap density is preferably 0.1 to 1 g/mL.

Specific examples of the carbon black used in the present invention include BLACKPEARLS 2000, 1300, 1000, 900, 800, 880, and 700, and VULCAN XC-72 (manufactured by Cabot Corporation), #3050B, #3150B, #3250B, #3750B, #3950B, #950, #650B, #970B, #850B, MA-600, MA-230, #4000, and #4010 (manufactured by Mitsubishi Chemical Corporation), CONDUCTEX SC, and RAVEN 8800, 8000, 7000, 5750, 5250, 3500, 2100, 2000, 1800, 1500, 1255, and 1250 (manufactured by Columbia Carbon Co.), Ketjen Black EC (manufactured by Akzo), and Ketjen Black EC (manufactured by Ketjen Black International Company Ltd.).

The carbon black may be subjected to any of a surface treatment with a dispersant, etc., grafting with a resin, or a partial surface graphitization. The carbon black may also be dispersed in a binder prior to addition to a coating solution. The carbon black can be preferably used in a range not exceeding 50 wt % relative to the above-mentioned inorganic powder, and in a range not exceeding 40 wt % relative to the total weight of the non-magnetic layer. The carbon black can be used singly or in a combination of different types thereof. The carbon black that can be used in the present invention can be referred to in, for example, the ‘Kabon Burakku Binran’ (Carbon Black Handbook) (edited by the Carbon Black Association of Japan).

It is also possible to add an organic powder to the non-magnetic layer depending on the intended purpose. Examples thereof include an acrylic styrene resin powder, a benzoguanamine resin powder, a melamine resin powder, and a phthalocyanine pigment, but a polyolefin resin powder, a polyester resin powder, a polyamide resin powder, a polyimide resin powder, and a polyfluoroethylene resin can also be used. Production methods such as those described in JP-A-62-18564 and JP-A-60-255827 can be used.

The binder, the lubricant, the dispersant, the additive, the solvent, the dispersion method, etc. for the non-magnetic layer may employ those used for the magnetic layer. In particular, with regard to the amount and type of binder, the additive, and the amount and type of dispersant, known techniques for the magnetic layer may be applied.

### III. Non-Magnetic Support

With regard to the non-magnetic support that can be used in the present invention, known biaxially stretched films such as polyethylene terephthalate, polyethylene naphthalate, polyamide, polyamideimide, and aromatic polyamide can be used. Polyethylene terephthalate, polyethylene naphthalate, and polyamide are preferred.

These supports can be subjected in advance to a corona discharge treatment, a plasma treatment, a treatment for enhancing adhesion, a thermal treatment, etc. The non-magnetic support that can be used in the present invention preferably has a surface roughness such that its center plane average surface roughness Ra is in the range of 3 to 10 nm for a cutoff value of 0.25 mm.

### IV. Smoothing Layer

The magnetic recording medium of the present invention may be provided with a smoothing layer. The smoothing layer referred to here is a layer for burying protrusions on the surface of the non-magnetic support; it is provided between the non-magnetic support and the magnetic layer when the magnetic recording medium is provided with the magnetic layer above the non-magnetic support, and it is provided between the non-magnetic support and the non-magnetic layer when the magnetic recording medium is provided with the non-magnetic layer and the magnetic layer in that order above the non-magnetic support.

The smoothing layer can be formed by curing a radiation curing type compound by exposure to radiation. The radiation curing type compound referred to here is a compound having the property of polymerizing or crosslinking when irradiated with radiation such as ultraviolet rays or an electron beam, thus increasing the molecular weight and carrying out curing.

### V. Backcoat Layer

In general, there is a strong requirement for magnetic tapes for recording computer data to have better repetitive transport properties than video tapes and audio tapes. In order to maintain such high storage stability, a backcoat layer can be provided on the surface of the non-magnetic support opposite to the surface where the non-magnetic layer and the magnetic layer are provided. As a coating solution for the backcoat layer, a binder and a particulate component such as an abrasive or an antistatic agent are dispersed in an organic solvent. As a granular component, various types of inorganic pigment or carbon black can be used. As the binder, a resin such as nitrocellulose, a phenoxy resin, a vinyl chloride resin, or a polyurethane can be used singly or in combination.

### VI. Layer Structure

In the constitution of the magnetic recording medium used in the present invention, the thickness of the non-magnetic support is preferably 3 to 80 μm. When the smoothing layer is provided between the non-magnetic support and the non-magnetic layer or the magnetic layer, the thickness of the smoothing layer is preferably 0.01 to 0.8 μm, and more preferably 0.02 to 0.6 μm. The thickness of the backcoat layer provided on the surface of the non-magnetic support opposite to the surface where the non-magnetic layer and the magnetic layer are provided is preferably 0.1 to 1.0 μm, and more preferably 0.2 to 0.8 μm.

The thickness of the magnetic layer is optimized according to the saturation magnetization and the head gap of the magnetic head and the bandwidth of the recording signal, but it is preferably 0.01 to 0.5 μm, more preferably 0.02 to 0.3 μm, and yet more preferably 0.03 to 0.2 μm. The percentage variation in thickness of the magnetic layer is preferably ±50% or less, and more preferably ±40% or less. The magnetic layer can be at least one layer, but it is also possible to provide two or more separate layers having different magnetic properties, and a known configuration for a multilayer magnetic layer can be employed.

The thickness of the non-magnetic layer is preferably 0.2 to 3.0 μm, more preferably 0.3 to 2.5 μm, and yet more preferably 0.4 to 2.0 μm. The non-magnetic layer of the magnetic recording medium of the present invention exhibits its effect if it is substantially non-magnetic, but even if it contains a small amount of a magnetic substance as an impurity or intentionally, if the effects of the present invention are exhibited the constitution can be considered to be substantially the same as that of the magnetic recording medium of the present invention. ‘Substantially the same’ referred to here means that the non-magnetic layer has a residual magnetic flux density of 10 mT (100 G) or less or a coercive force of 7.96 kA/m (100 Oe) or less, and preferably has no residual magnetic flux density and no coercive force.

### VII. Production Method

A process for producing a magnetic layer coating solution for the magnetic recording medium used in the present invention comprises at least a kneading step, a dispersing step and, optionally, a blending step that is carried out prior to and/or subsequent to the above-mentioned steps. Each of these steps may be composed of two or more separate stages. All materials, including the ferromagnetic hexagonal ferrite powder, the ferromagnetic metal powder, the non-magnetic powder, the binder, the carbon black, the abrasive, the antistatic agent, the lubricant, and the solvent used in the present invention may be added in any step from the beginning or during the course of the step. The addition of each material may be divided across two or more steps. For example, a polyurethane can be divided and added in a kneading step, a dispersing step, and a blending step for adjusting the viscosity after dispersion. To attain the object of the present invention, a conventionally known production technique may be employed as a part of the steps. In the kneading step, it is preferable to use a powerful kneading machine such as an open kneader, a continuous kneader, a pressure kneader, or an extruder. When a kneader is used, all or a part of the binder (preferably 30 wt % or above of the entire binder) is preferably kneaded with the magnetic powder or the non-magnetic powder at 15 to 500 parts by weight of the binder relative to 100 parts by weight of the magnetic substance. Details of these kneading treatments are described in JP-A-1-106338 and JP-A-1-79274. For the dispersion of the magnetic layer solution and a non-magnetic layer solution, glass beads can be used. As such glass beads, a dispersing medium having a high specific gravity such as zirconia beads, titania beads, or steel beads is suitably used. An optimal particle size and packing density of these dispersing media is used. A known disperser can be used.

The process for producing the magnetic recording medium of the present invention includes, for example, coating the surface of a moving non-magnetic support with a magnetic layer coating solution so as to give a predetermined coating thickness. A plurality of magnetic layer coating solutions can be applied successively or simultaneously in multilayer coating, and a lower magnetic layer coating solution and an upper magnetic layer coating solution can also be applied successively or simultaneously in multilayer coating. As coating equipment for applying the above-mentioned magnetic layer coating solution or the lower magnetic layer coating solution, an air doctor coater, a blade coater, a rod coater, an extrusion coater, an air knife coater, a squeegee coater, a dip coater, a reverse roll coater, a transfer roll coater, a gravure coater, a kiss coater, a cast coater, a spray coater, a spin coater, etc. can be used. With regard to these, for example, ‘Saishin Kotingu Gijutsu’ (Latest Coating Technology) (May 31, 1983) published by Sogo Gijutsu Center can be referred to.

In the case of a magnetic tape, the coated layer of the magnetic layer coating solution is subjected to a magnetic field alignment treatment in which the ferromagnetic powder contained in the coated layer of the magnetic layer coating solution is aligned in the longitudinal direction using a cobalt magnet or a solenoid. In the case of a disk, although sufficient isotropic alignment can sometimes be obtained without using an alignment device, it is preferable to employ a known random alignment device such as, for example, arranging obliquely alternating cobalt magnets or applying an alternating magnetic field with a solenoid. The isotropic alignment referred to here means that, in the case of a ferromagnetic metal powder, in general, in-plane two-dimensional random is preferable, but it can be three-dimensional random by introducing a vertical component. In the case of a ferromagnetic hexagonal ferrite powder, in general, it tends to be in-plane and vertical three-dimensional random, but in-plane two-dimensional random is also possible. By using a known method such as magnets having different poles facing each other so as to make vertical alignment, circumferentially isotropic magnetic properties can be introduced. In particular, when carrying out high density recording, vertical alignment is preferable. Furthermore, circumferential alignment may be employed using spin coating.

It is preferable for the drying position for the coating to be controlled by controlling the drying temperature and blowing rate and the coating speed; it is preferable for the coating speed to be 20 to 1,000 m/min and the temperature of drying air to be 60° C. or higher, and an appropriate level of pre-drying may be carried out prior to entering a magnet zone.

After drying is carried out, the coated layer is subjected to a surface smoothing treatment. The surface smoothing treatment employs, for example, super calender rolls, etc. By carrying out the surface smoothing treatment, cavities formed by removal of the solvent during drying are eliminated, thereby increasing the packing ratio of the ferromagnetic powder in the magnetic layer, and a magnetic recording medium having high electromagnetic conversion characteristics can thus be obtained.

With regard to calendering rolls, rolls of a heat-resistant plastic such as epoxy, polyimide, polyamide, or polyamideimide are used. It is also possible to carry out a treatment with metal rolls. The magnetic recording medium of the present invention preferably has a surface center plane average roughness in the range of 0.1 to 4.0 nm for a cutoff value of 0.25 mm, and more preferably 0.5 to 3.0 nm, which is extremely smooth. As a method therefor, a magnetic layer formed by selecting a specific ferromagnetic powder and binder as described above is subjected to the above-mentioned calendering treatment. With regard to calendering conditions, the calender roll temperature is preferably in the range of 60° C. to 100° C., more preferably in the range of 70° C. to 100° C., and particularly preferably in the range of 80° C. to 100° C., and the pressure is preferably in the range of 100 to 500 kg/cm, more preferably in the range of 200 to 450 kg/cm, and particularly preferably in the range of 300 to 400 kg/cm.

As thermal shrinkage reducing means, there is a method in which a web is thermally treated while handling it with low tension, and a method (thermal treatment) involving thermal treatment of a tape when it is in a layered configuration such as in bulk or installed in a cassette, and either can be used. In the former method, the effect of the imprint of protrusions of the surface of the backcoat layer is small, but the thermal shrinkage cannot be greatly reduced. On the other hand, the latter thermal treatment can improve the thermal shrinkage greatly, but since the effect of the imprint of protrusions of the surface of the backcoat layer is strong, the surface of the magnetic layer is roughened, and this causes the output to decrease and the noise to increase. In particular, a high output and low noise magnetic recording medium can be provided for the magnetic recording medium accompanying the thermal treatment. The magnetic recording medium thus obtained can be cut to a desired size using a cutter, a stamper, etc. before use.

### VIII. Physical Properties

The saturation magnetic flux density of the magnetic layer of the magnetic recording medium of the present invention is preferably 100 to 300 mT (1,000 to 3,000 G). The coercive force (Hc) of the magnetic layer is preferably 143.3 to 318.4 kA/m (1,800 to 4,000 Oe), and more preferably 159.2 to 278.6 kA/m (2,000 to 3,500 Oe). It is preferable for the coercive force distribution to be narrow, and the SFD and SFDr are preferably 0.6 or less, and more preferably 0.2 or less.

The coefficient of friction, with respect to a head, of the magnetic recording medium used in the present invention is preferably 0.5 or less at a temperature of −10° C. to 40° C. and a humidity of 0% to 95%, and more preferably 0.3 or less. The electrostatic potential is preferably −500 V to +500 V. The modulus of elasticity of the magnetic layer at an elongation of 0.5% is preferably 0.98 to 19.6 GPa (100 to 2,000 kg/mm²) in each direction within the plane, and the breaking strength is preferably 98 to 686 MPa (10 to 70 kg/mm²); the modulus of elasticity of the magnetic recording medium is preferably 0.98 to 14.7 GPa (100 to 1,500 kg/mm²) in each direction within the plane, the residual elongation is preferably 0.5% or less, and the thermal shrinkage at any temperature up to and including 100° C. is preferably 1% or less, more preferably 0.5% or less, and most preferably 0.1% or less.

The glass transition temperature of the magnetic layer (the maximum point of the loss modulus in a dynamic viscoelasticity measurement at 110 Hz) is preferably 50° C. to 180° C., and that of the non-magnetic layer is preferably 0° C. to 180° C. The loss modulus is preferably in the range of 1×10⁷ to 8×10⁸ Pa (1×10⁸ to 8×10⁹ dyne/cm²), and the loss tangent is preferably 0.2 or less. When the loss tangent is 0.2 or less, the problem of tackiness is suppressed. These thermal properties and mechanical properties are preferably substantially identical to within 10% in each direction in the plane of the medium.

Residual solvent in the magnetic layer is preferably 100 mg/m² or less, and more preferably 10 mg/m² or less. The porosity of the coating layer is preferably 30 vol % or less for both the non-magnetic layer and the magnetic layer, and more preferably 20 vol % or less. In order to achieve a high output, the porosity is preferably low, but there are cases in which a certain value should be maintained depending on the intended purpose. For example, in the case of disk media where repetitive use is considered to be important, a high porosity is often preferable from the point of view of storage stability.

The center plane surface roughness Ra of the magnetic layer is preferably 4.0 nm or less, more preferably 3.0 nm or less, and yet more preferably 2.0 nm or less, when measured using a TOPO-3D digital optical profiler (manufactured by Wyko Corporation). The maximum height SRₘₐₓ of the magnetic layer is preferably 0.5 μm or less, the ten-point average roughness SRz is 0.3 μm or less, the center plane peak height SRp is 0.3 μm or less, the center plane valley depth SRv is 0.3 μm or less, the center plane area factor SSr is 20% to 80%, and the average wavelength Sλa is 5 to 300 λm. It is possible to set the number of surface protrusions on the magnetic layer having a size of 0.01 to 1 μm at any level in the range of 0 to 2,000 protrusions per 100 μm, and by so doing the electromagnetic conversion characteristics and the coefficient of friction can be optimized, which is preferable. They can be controlled easily by controlling the surface properties of the support by means of a filler, the particle size and the amount of a powder added to the magnetic layer, and the shape of the roll surface in the calendering process. The curl is preferably within ±3 mm.

When the magnetic recording medium of the present invention has a non-magnetic layer and a magnetic layer, it can easily be anticipated that the physical properties of the non-magnetic layer and the magnetic layer can be varied according to the intended purpose. For example, the elastic modulus of the magnetic layer can be made high, thereby improving the storage stability, and at the same time the elastic modulus of the non-magnetic layer can be made lower than that of the magnetic layer, thereby improving the head contact of the magnetic recording medium.

A head used for playback of signals recorded magnetically on the magnetic recording medium of the present invention is not particularly limited, but an MR head is preferably used. When an MR head is used for playback of the magnetic recording medium of the present invention, the MR head is not particularly limited and, for example, a GMR head or a TMR head can be used. A head used for magnetic recording is not particularly limited, but it is preferable for the saturation magnetization to be 1.0 T or more, and preferably 1.5 T or more.

In accordance with the present invention, there can be provided a carbonic acid ester that can be used suitably as a lubricant. Moreover, in accordance with the present invention, a magnetic recording medium that employs the carbonic acid ester and that has excellent durability in a low temperature environment and excellent storage stability can be provided.

### EXAMPLES

The present invention is explained more specifically below by reference to Examples, but the present invention should not be construed as being limited to the Examples. ‘Parts’ in the Examples means ‘parts by weight’ unless otherwise specified.

### Example 1

### Lubricant A Synthetic Example

A flask was charged with 86 parts of 1-tetradecanol, 264 parts of hexane, and 35 parts of pyridine, and cooled while stirring. 42 parts of 2-ethylhexyl chloroformate was further added dropwise to this flask while cooling and stirring over 2 hours. While further stirring the interior of the flask, it was taken to room temperature and allowed to stand for 6 hours. Water was added to this reaction mixture, the mixture was stirred and then left to stand, and the aqueous layer was run off using a separatory funnel. Methanol was added, the mixture was stirred and then left to stand, and the methanol phase was separated; this operation was repeated three times. The remaining hexane solution was concentrated under vacuum, and 135 parts of crude lubricant A, which was a colorless transparent liquid, was obtained.

This liquid was diluted 2 times with hexane and purified by means of column chromatography, and the hexane solution was concentrated under vacuum, thus giving 77 parts of lubricant A.

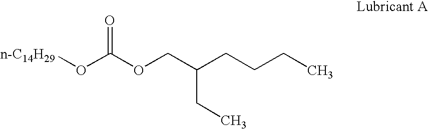

### Examples 2 and 3

### Lubricants B and C Synthetic Examples

The procedure of Example 1 was repeated except that the 1-tetradecanol of Example 1 was changed to 1-hexanol or 1-dodecanol, and lubricants B and C were obtained.

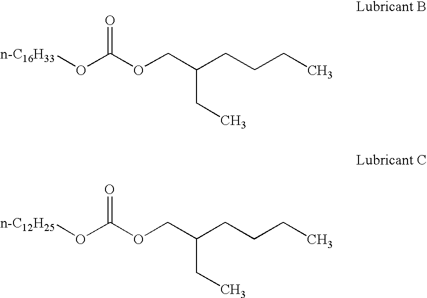

### Examples 4 and 5

### Lubricants D and E Synthetic Examples

The procedure of Example 1 was repeated except that the 2-ethylhexyl chloroformate of Example 1 was changed to 2-methylpropyl chloroformate or 2-methylbutyl chloroformate, and lubricants D and E were obtained.

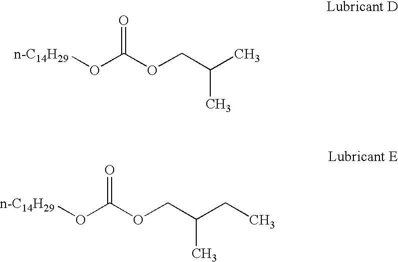

### Examples 6 and 7

### Lubricants F and G Synthetic Examples

The procedure of Example 1 was repeated except that the 1-tetradecanol of Example 1 was changed to 1-dedecanol, and the 2-ethylhexyl chloroformate was changed to 2-methylpropyl chloroformate or 2-methylbutyl chloroformate, and lubricants F and G were obtained.

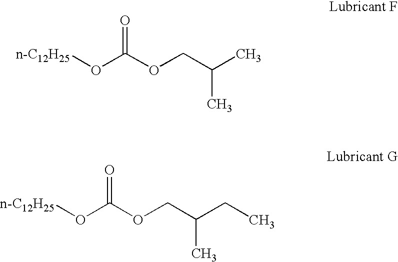

### Comparative Examples 1 and 2

### Lubricants H and I Synthetic Examples

The procedure of Example 1 was repeated except that the 1-tetradecanol of Example 1 was changed to 1-octadecanol, and the 2-ethylhexyl chloroformate was changed to butyl chloroformate or methyl chloroformate, and lubricants H and I were obtained.

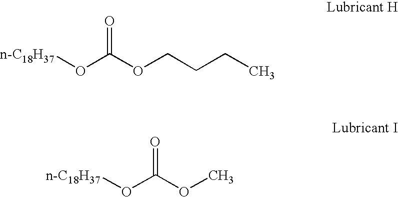

### Comparative Example 3

### Lubricant J Synthetic Example

The procedure of Example 1 was repeated except that the 1-tetradecanol of Example 1 was changed to 1-octadecanol, and lubricant J was obtained.

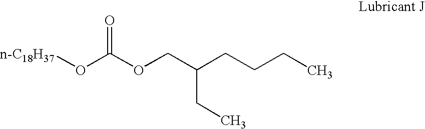

### Comparative Example 4

### Lubricant K Synthetic Example

The procedure of Example 1 was repeated except that the 1-tetradecanol of Example 1 was changed to 1-octadecanol, and the 2-ethylhexyl chloroformate was changed to 2-ethylhexanoyl chloride, and lubricant K was obtained.

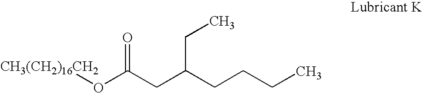

### Measurement Method

Melting point was measured for lubricants A and B and lubricants H to K by means of DSC (rate of change of temperature −5° C./m in). It was −23° C. for lubricant A and −3° C. for lubricant B.

For lubricants E to G, they were allowed to stand in a constant temperature chamber set at −5° C. for 24 hours and shaken in the constant temperature chamber for 5 minutes, thus checking that they maintained a liquid state.

### Examples 8 to 14 and Comparative Examples 5 to 8

Preparation of upper layer magnetic solution

| | | |
|---|---|---|
| 100 parts of a ferromagnetic metal powder (Co/Fe = 30 atom %, Hc: | 2 | parts |
| 2,350 Oe (187 kA/m), S_{BET}: 55 m²/g, surface treated with Al₂O₃, SiO₂, and Y₂O₃, | | |
| average major axis length: 50 nm, average acicular ratio: 7, σs: 120 A · m²/kg) | | |
| was ground in an open kneader for 10 minutes, subsequently | | |
| carbon black (average particle size 80 nm) | | |
| a vinyl chloride resin (MR-110, manufactured by Nippon Zeon Corporation) | 10 | parts |
| a polyester polyurethane (UR8300, manufactured by Toyobo Co., Ltd.) | 6 | parts (solids |
| | | content), and |
| methyl ethyl ketone/cyclohexanone = 1/1 | 60 | parts |
| were added thereto, and the mixture was kneaded for 60 minutes. To this | | |
| mixture, | | |
| methyl ethyl ketone/cyclohexanone = 1/1 | 200 | parts |
| was added over 6 hours while operating the open kneader. Subsequently, | 20 | parts |
| an α-Al₂O₃ dispersion (solids content 40%) | | |
| was added thereto, and the mixture was dispersed in a sand grinder for 120 | | |
| minutes. Furthermore, | | |
| a polyisocyanate | 4 | parts (solids |
| (Coronate 3041, manufactured by Nippon Polyurethane Industry Co., Ltd.) | | content) |
| stearic acid | 1 | part |
| lubricant described in table | 2 | parts |
| stearamide | 0.2 | parts, and |
| toluene | 50 | parts |
| were added thereto, and the mixture was stirred and mixed for 20 minutes. | | |

Following this, the mixture was filtered using a filter having an average pore size of 1 μm to give a magnetic coating solution.

Preparation of lower layer non-magnetic solution

| | | |
|---|---|---|
| 85 parts of titanium oxide (average particle size 0.035 μm, rutile crystal | | |
| type, TiO₂ content 90% or greater, surface treated with alumina, S_{BET} 35 to 42 m²/g, | | |
| true specific gravity 4.1, pH 6.5 to 8.0) and 15 parts of carbon black | | |
| (Ketjen black EC, manufactured by Nippon EC) were ground in an open | | |
| kneader for 10 minutes, subsequently | | |
| a vinyl chloride copolymer (MR-110, manufactured by Nippon Zeon | 17 | parts |
| Corporation) | | |
| a sulfonic acid-containing polyurethane resin (UR8200, manufactured by | 10 | parts (solids |
| Toyobo Co., Ltd.) | | content), and |
| cyclohexanone | 60 | parts |
| were added thereto, and the mixture was kneaded for 60 minutes. | | |
| Subsequently, | | |
| methyl ethyl ketone/cyclohexanone = 6/4 | 200 | parts |
| was added thereto, and the mixture was dispersed in a sand mill for 120 | | |
| minutes. To this were added | | |
| a polyisocyanate (Coronate 3041, manufactured by Nippon Polyurethane | 5 | parts (solids |
| Industry Co., Ltd.) | | content) |
| stearic acid | 1 | part |
| lubricant described in table | 2 | parts |
| oleic acid | 1 | part, and |
| methyl ethyl ketone | 50 | parts, |
| and the mixture was stirred and mixed for 20 minutes, then filtered using a filter | | |
| having an average pore size of 1 μm to give a non-magnetic coating solution. | | |

The surface of a 62 μm thick polyethylene terephthalate support was coated with the non-magnetic coating solution thus obtained and, immediately after that, with the magnetic coating solution by simultaneous multilayer coating so that the dry thicknesses thereof were 1.5 μm and 0.2 μm respectively. Before the magnetic coating solution had dried, it was subjected to magnetic field alignment using a 5,000 G Co magnet and a 4,000 G solenoid magnet, and after removing the solvent by drying, it was subjected to a calender treatment employing a metal roll-metal roll-metal roll-metal roll-metal roll-metal roll-metal roll combination (speed 100 m/min, line pressure 300 kg/cm, temperature 90° C.) and then slit to a width of ½ inch (=12.65 mm).

### Measurement Methods

### 1. Durability and Storage Stability

The sliding durability of the tape was measured as follows. That is, the tape was made to slide at a sliding speed of 2 m/sec repeatedly for 10,000 passes under an environment of 5° C. and 80% RH with the magnetic layer surface in contact with an AlTiC cylindrical rod at a load of 100 g (T1), and tape damage was then evaluated. Evaluation was carried out using the rankings below.

Furthermore, 600 m of tape was stored at 60° C. and 90% RH for 6 months while wound on a reel for an LTO-G3 cartridge. The tape after storage was evaluated in the same manner.

Excellent: slightly scratched, but area without scratches was larger.
Good: area with scratches was larger than area without scratches.
Poor: magnetic layer completely peeled off.

Evaluation results for Examples 8 to 14 and Comparative Examples 5 to 8 are given in Table 1 below.

TABLE 1

| | Lubricant | | | | | | Durability and storage stability | |
|---|---|---|---|---|---|---|---|---|
| | | | Molecular structure | | Melting | | After sliding | After storage |
| | No. | Type | R¹ | R² | point (° C.) | | durability test | at 60° C. 90% |
| Ex. 8 | A | Carbonic | 2-Ethylhexyl | Tetradecyl | −23° | C. | Excellent | Excellent |
| | | acid ester | | | | | | |
| Ex. 9 | B | Carbonic | 2-Ethylhexyl | Hexadecyl | −3° | C. | Excellent | Excellent |
| | | acid ester | | | | | | |
| Ex. 10 | C | Carbonic | 2-Ethylhexyl | Dodecyl | 0° | C.≧ | Good | Good |
| | | acid ester | | | | | | |
| Ex. 11 | D | Carbonic | 2-Methylpropyl | Tetradecyl | 0° | C.≧ | Good | Good |
| | | acid ester | | | | | | |
| Ex. 12 | E | Carbonic | 2-Methylbutyl | Tetradecyl | 0° | C.≧ | Excellent | Excellent |
| | | acid ester | | | | | | |
| Ex. 13 | F | Carbonic | 2-Methylpropyl | Dodecyl | 0° | C.≧ | Good | Good |
| | | acid ester | | | | | | |
| Ex. 14 | G | Carbonic | 2-Methylbutyl | Dodecyl | 0° | C.≧ | Excellent | Excellent |
| | | acid ester | | | | | | |
| Comp. Ex. 5 | H | Carbonic | Butyl | Octadecyl | 53° | C. | Poor | Poor |
| | | acid ester | | | | | | |
| Comp. Ex. 6 | I | Carbonic | Methyl | Octadecyl | 46° | C. | Poor | Poor |
| | | acid ester | | | | | | |
| Comp. Ex. 7 | J | Carbonic | 2-Ethylhexyl | Octadecyl | 13° | C. | Poor | Good |
| | | acid ester | | | | | | |
| Comp. Ex. 8 | K | Fatty acid | 2-Ethylhexyl | Octadecyl | 9° | C. | Good | Poor |
| | | ester | | | | | | |

What is claimed is:

1. A carbonic acid ester represented by Formula (1) and having a melting point of no greater than 0° C., 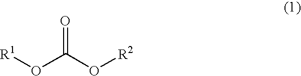
(in Formula (1), R¹ and R² independently denote a saturated hydrocarbon group, R¹ is a branched chain, and R² is a straight or branched chain).

2. The carbonic acid ester according to claim 1, wherein the sum of the number of carbons of R¹ and R² is at least 12 but no greater than 50.

3. The carbonic acid ester according to claim 1, wherein the number of carbons of R¹ is at least 3 but no greater than 12.

4. The carbonic acid ester according to claim 1, wherein R¹ has a structure in which it is branched at the β-position.

5. The carbonic acid ester according to claim 1, wherein R¹ is selected from the group consisting of a 2-methylpropyl group, a 2-methylbutyl group, and a 2-ethylhexyl group.

6. The carbonic acid ester according to claim 1, wherein R² is a straight-chain structure having at least 12 but no greater than 16 carbon atoms.

7. The carbonic acid ester according to claim 1, wherein R² is selected from the group consisting of an n-dodecyl group, an n-tetradecyl group, and an n-hexadecyl group.

8. A magnetic recording medium comprising:
a non-magnetic support and, above the support;
at least one magnetic layer comprising a ferromagnetic powder dispersed in a binder,
the magnetic layer comprising the carbonic acid ester according to claim 1.

9. The magnetic recording medium according to claim 8, wherein the magnetic layer comprises at least 0.1 wt % but no greater than 5 wt % of the carbonic acid ester.

10. The magnetic recording medium according to claim 8, wherein the non-magnetic support has a thickness of at least 3 μm but no greater than 80 μm.

11. The magnetic recording medium according to claim 8, wherein the magnetic layer has a thickness of at least 0.01 μm but no greater than 0.5 μm.

12. A magnetic recording medium comprising:
a support and, above the support;
a non-magnetic layer comprising a non-magnetic powder dispersed in a binder and, above the non-magnetic layer;
at least one magnetic layer comprising a ferromagnetic powder dispersed in a binder,
the non-magnetic layer and/or the magnetic layer comprising the carbonic acid ester according to claim 1.

13. The magnetic recording medium according to claim 12, wherein the non-magnetic layer comprises at least 0.1 wt % but no greater than 5 wt % of the carbonic acid ester.

14. The magnetic recording medium according to claim 12, wherein the non-magnetic layer has a thickness of at least 0.2 μm but no greater than 3.0 μm.
